(19) United States
(12) Patent Application Publication
(10) Pub. No.: US 2025/0305942 A1
(43) Pub. Date: Oct. 2, 2025

(54) SELF-SUPPORTING SEGMENTED BLADDERS FOR REMOTE SENSING

(71) Applicant: Mission Support and Test Services, LLC, Las Vegas, NV (US)

(72) Inventors: John Di Benedetto, Santa Barbara, CA (US); Ian Bortins, Santa Barbara, CA (US)

(21) Appl. No.: 19/093,874

(22) Filed: Mar. 28, 2025

Related U.S. Application Data
(60) Provisional application No. 63/572,134, filed on Mar. 29, 2024.

(51) Int. Cl. G01N 21/31; G01N 21/03; G01N 33/00

(52) U.S. Cl. CPC G01N 21/31; G01N 21/03; G01N 33/0027

(57) ABSTRACT

Provided are a system and method for self-supporting segmented gas containers for remote sensing. Embodiments include a system for containing a type of gas to allow optical detection of the gas in a spectral region of detection defined by the type of gas, including: a top layer of material; a bottom layer of material, wherein the top layer is sealed to the bottom layer forming a bladder with a perimeter seam and forming a cavity; ribbing between the top layer of material and the bottom layer of material defining segments of the cavity; and a frame, where the frame supports the bladder.

### CROSS-REFERENCE TO RELATED APPLICATIONS

This application claims priority to U.S. Provisional Patent Application Ser. No. 63/572,134, filed on Mar. 29, 2024, the contents of which are herein incorporated by reference in their entirety.

### STATEMENT REGARDING FEDERAL RIGHTS

This invention mas made with government support under Contract No. DE-NA0003624 effective Dec. 1, 2017, updated to include modifications through 0177, and was awarded by the U.S. Department of Energy, National Nuclear Security Administration. The government has certain rights in the invention.

### TECHNOLOGICAL FIELD

Embodiments of the present disclosure relate generally to optical gas target systems, and more particularly to self-supporting segmented gas containers for remote sensing.

### BACKGROUND

Many systems have been developed for the remote sensing of gases to detect a type of gas and/or factors concerning the gas, such as a concentration or quantity of the gas. These systems may be operated from a remote location, such as from an aircraft flying over the gas or even from a satellite flying overhead. For example, such detection systems may be used to detect leaks from chemical plants or pipelines, to detect chemical weapons, to detect greenhouse gases, to detect gas leaks in man-made disasters, etc.

The gas detection systems may use solar illumination or thermal radiance to image optical features of the gas, which are measured by a sensor or imaging device included in the gas detection system. Some of the gas detection systems employ one or more lasers with light from the laser directed at the gas, with the sensor measuring a reflected signal. For example, the sensor can be used to measure absorptive or emissive properties of the gas, from which the type of gas and other factors may be determined from known techniques. However, the amount of gas can be difficult to quantify in an open-air plume of gas. These systems use the temperature of the gas and the temperature of the background in making the determinations of the gas, where the imager sees an absorption or an emission depending on the temperature differences.

The sensor may be an infrared (IR) or thermal hyperspectral image sensor for example. Such sensors are often used in remote environmental monitoring of gaseous plumes in the environment. Calibration of such remote sensing systems can be difficult, because gases released into the environment may be difficult to quantify due to drifting of the gas based on environmental conditions such as wind, varying temperatures and other factors, as well as the dangers of releasing a gas, which may be toxic, into the environment

### BRIEF SUMMARY

A system, apparatus, and method are provided herein for optical gas target systems, and more particularly to self-supporting segmented gas containers for remote sensing. Embodiments include a system for containing a type of gas to allow optical detection of the gas in a spectral region of detection defined by the type of gas, including: a top layer of material; a bottom layer of material, wherein the top layer is sealed to the bottom layer forming a bladder with a perimeter seam and forming a cavity; ribbing between the top layer of material and the bottom layer of material defining segments of the cavity; and a frame, where the frame supports the bladder.

The system of an example embodiment further includes a reinforcing strip substantially covering the perimeter seam. The system of some embodiments further includes one or more grommets within the reinforcing strip, wherein the bladder is attached to the frame using the one or more grommets. According to certain embodiments the segments of the cavity are of substantially similar height. According to some embodiments the bladder comprises a rectangular shape. The bladder of an example embodiments includes a hexagonal shape.

According to some embodiments at least one of the top layer of material and the bottom layer of material are substantially optically transparent material of at least 80% transparent in a spectral region of detection for the system. According to certain embodiments at least one of the top layer of material and the bottom layer of material is sufficiently pure to decrease absorptions in a spectral region of detection for the system to less than between 5% and 20%. The at least one of the top layer of material and the bottom layer of material of an example embodiment is polyethylene. According to certain embodiments bladder is formed on a roll, and wherein the bladder is cut to a user-defined length at a cut line, and wherein the cut line is sealed to close the cavity.

Embodiments provided herein include a method of containing a type of gas to allow optical detection of the gas in a spectral region of detection defined by the type of gas, the method including: filling a bladder with the gas, wherein the bladder includes: a top layer of material; a bottom layer of material, wherein the top layer is sealed to the bottom layer forming a bladder with a perimeter seam and forming a cavity; ribbing between the top layer of material and the bottom layer of material defining segments of the cavity; and supporting the bladder on a frame.

According to some embodiments the bladder includes a reinforcing strip substantially covering the perimeter seam. The reinforcing strip of an example embodiment further includes one or more grommets within the reinforcing strip, wherein supporting the bladder on the frame comprises attaching the bladder to the frame using the one or more grommets. According to certain embodiments the segments of the cavity are of substantially similar height. The bladder of some embodiments includes a rectangular shape. The bladder of some embodiments includes a hexagonal shape.

According to some embodiments at least one of the top layer of material and the bottom layer of material are substantially optically transparent material of at least 80% transparent in the spectral region of detection. According to certain embodiments at least one of the top layer of material and the bottom layer of material is sufficiently pure to decrease absorptions in the spectral region of detection to less than between 5% and 20%. The at least one of the top layer of material and the bottom layer of material of an example embodiment is polyethylene. The method of some embodiments further includes: forming the bladder on a roll; cutting the bladder to a user-defined length at a cut line; and sealing the bladder at the cut line to form the cavity.

### BRIEF DESCRIPTION OF DRAWINGS

Having thus described certain embodiments of the present disclosure in general terms, reference will now be made to the accompanying drawings, which are not necessarily drawn to scale, and wherein:

FIG. 1 illustrates an example embodiment of a top view of a bladder having first geometry of a hexagon shape according to an example embodiment of the present disclosure;

FIG. 2 illustrates a second example embodiment of a top view of a bladder and an end view according to an example embodiment of the present disclosure;

FIG. 3 illustrates an example bladder that can be fabricated using a variable length long bladder according to an example embodiment of the present disclosure;

FIG. 4 illustrates a bladder having a hexagon shape suspended within a lightweight frame according to an example embodiment of the present disclosure;

FIG. 5 illustrates light rays from the sun passing through the gas container bladder according to an example embodiment of the present disclosure;

FIG. 6 illustrates the light rays that first reflect off the ground, then pass through the gas container bladder according to an example embodiment of the present disclosure;

FIG. 7 illustrates an alternative deployment configuration where the gas container bladder is placed on the ground and light passes through the bladder twice according to an example embodiment of the present disclosure;

FIG. 8 illustrates another alternative deployment configuration where the gas container bladder is placed at an angle relative to the light rays and used to cast a spectral feature on to a second structure, which may be a wall according to an example embodiment of the present disclosure;

FIG. 9 illustrates a gas container bladder and its support structure frame used to detect changes in absorption according to an example embodiment of the present disclosure;

FIG. 10 illustrates a gas container bladder and its support structure frame used to detect changes in emissivity according to an example embodiment of the present disclosure;

FIG. 11 illustrates alternative arrangements for the gas container bladders including a spectral radiant sources positioned adjacent to the gas container bladders according to an example embodiment of the present disclosure;

FIG. 12 illustrates the bladder geometry for 28 targets or bladders that are each two meters by two meters according to an example embodiment of the present disclosure; and

FIG. 13 illustrates an embodiment using hexagonal bladders according to an example embodiment of the present disclosure.

### DETAILED DESCRIPTION

The present disclosure now will be described more fully hereinafter with reference to the accompanying drawings, in which some, but not all embodiments are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

Provided herein is a system and corresponding methods for containing a type of gas to allow optical detection of the gas in a spectral region of detection defined by the type of gas. The system includes a gas container or “bladder” containing the gas, the gas container formed from a material substantially optically transparent in the spectral region of detection defined by the type of gas. Embodiments include a segmented bladder that holds its shape and volume with internal pressure. The bladder of an example embodiment gains stability and size (depth) control by filling to a specific low pressure that creates rigidity. While any surface geometry can be created, rectilinear and hexagon shapes are proposed for fielding large numbers of individual targets in an array.

The container, described as a bag or bladder is an optical container and these terms will be used often as the container. A bladder of an embodiment defines a shape and volume prescribed not only by the outer bladder material, but also by ribbing or internal structure of the bladder which defines chambers of the bladder, and the bladder is inflated to dimensions specified by the bladder material and internal structure. The bladder may be any shape and dimension and may have a top layer secured to a bottom layer at the edges with an internal structure of ribbing that limits the separation of the top layer and the bottom layer between the edges or perimeter. Optionally, the ribbing or internal structure can be formed by attaching a portion of the top layer to a portion of the bottom layer, such as along a seam, to form chambers. Forming the bladder as described herein provides structure and rigidity to the bladder without requiring a frame, thus reducing cost, weight, and complexity of the bladder as compared to prior iterations.

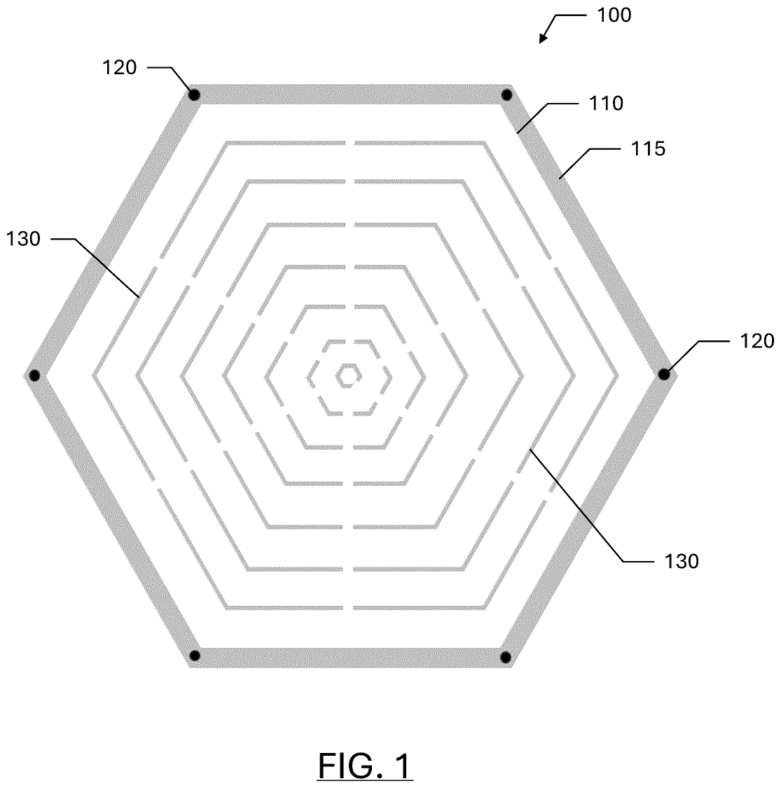

The bladder of an example embodiment is configured similarly to a raft with connected chambers. FIG. 1 illustrates an example embodiment of a top view of a bladder 100 having first geometry of a hexagon shape. The bladder 100 includes a perimeter seam 110 between a top layer and a bottom layer. The perimeter seam 110 may include a reinforcing strip 115, such as a nylon webbing, which can relieve strain on the bladder and the reinforcing strip can, in some embodiments, include grommets 120. The grommets 120 can be placed at each vertex, for example. The bladder includes ribbing 130 to define segments of the bladder there between and to limit travel of the top layer and bottom layer away from one another. Without the ribbing 130, the bladder 100 would inflate to take a shape with both the top and bottom layers being domed with a substantial height between a center of the top layer and the bottom layer. The bladder of an example embodiment in a hexagonal configuration as shown in FIG. 1 may have a width of about two meters, or an apothem of about one meter.

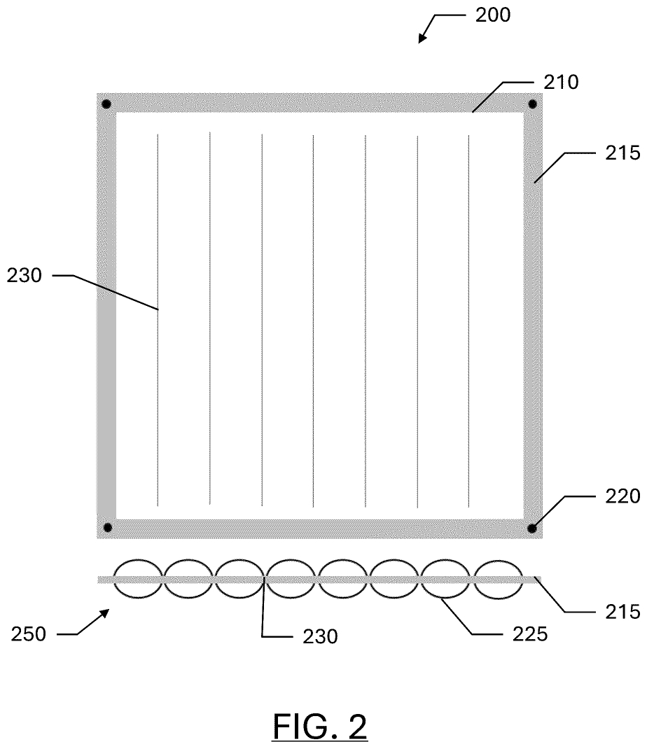

FIG. 2 illustrates a second example embodiment of a top view of a bladder 200 and an end view 250. As shown, the bladder 200 defines a square shape having a perimeter seam 210 and a reinforcing strip 215 with grommets 220 at each vertex. Also depicted is the ribbing 230. The end view 250 illustrates the reinforcing strip 215, where the ribbing defines the segments 225 of the bladder. As shown, each segment 225 of the bladder is of a substantially similar height where substantially similar is defined herein as within about 15% of each other.

Embodiments of the bladders described herein hold their shape and volume in response to internal pressure. Embodiments can be employed in remote sensing systems under test that have very large individual pixels in the form of bladders. These relatively large structures are lightweight and can thus be elevated with ease for placement on poles, for example or suspended with other means. Embodiments have stability and size controlled by filling the bladder to a specific low pressure that creates rigidity. While any surface geometry can be created, rectilinear and hexagonal shapes are particularly desirable for fielding large numbers of individual targets in an array as they nest well together. The bladders of example embodiments are lightweight and can be supported with a frame, such as a tubular aluminum frame. However, the frame does not define the bladder shape, but merely holds the bladder in position through adverse environmental conditions.

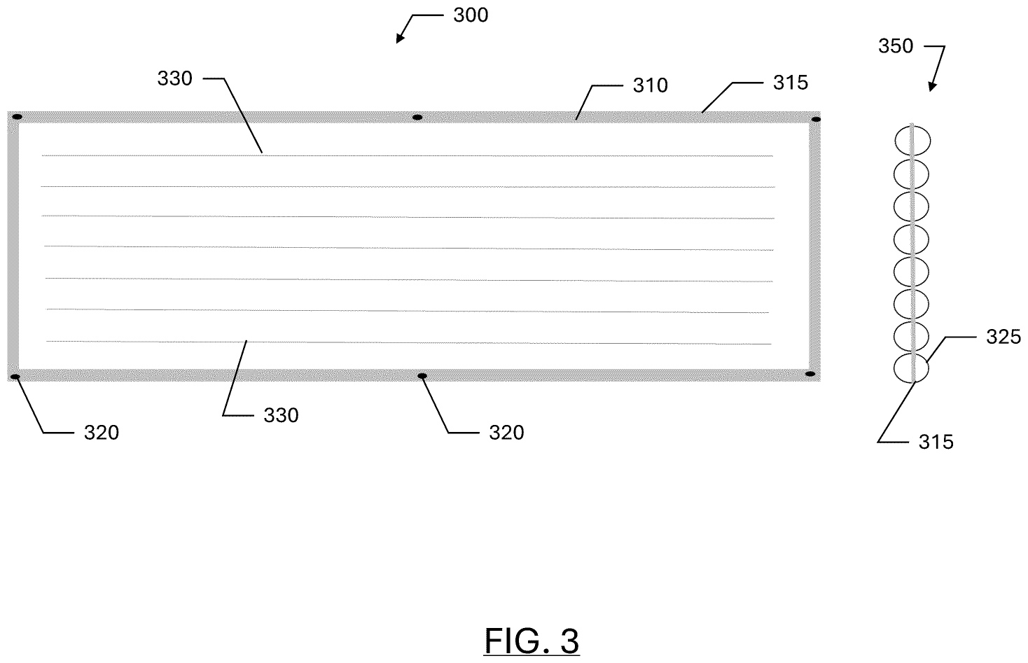

FIG. 3 illustrates an example bladder 300 that can be fabricated using a variable length long bladder. The material of the bladder 300 can be on a roll and the length set by the exposed length of the bladder. The material can be cut at any length and seams welded (e.g., via heat or ultrasonic welding) where the material is cut. The reinforcing strip 315 can be applied to the cut end after cutting while the reinforcing strip along the sides can be continuous. As the long bladder 300 lacks vertices along the length, grommets 320 can be included along a length of the bladder as well as at the vertices. The ribbing 330 can be perforated or include pass throughs or apertures between segments 325 to enable air to pass between segments even when a scam is created along the length of the material. The end view 350 illustrates the segments 325 and the reinforcing strip 315 which covers the perimeter seam 310.

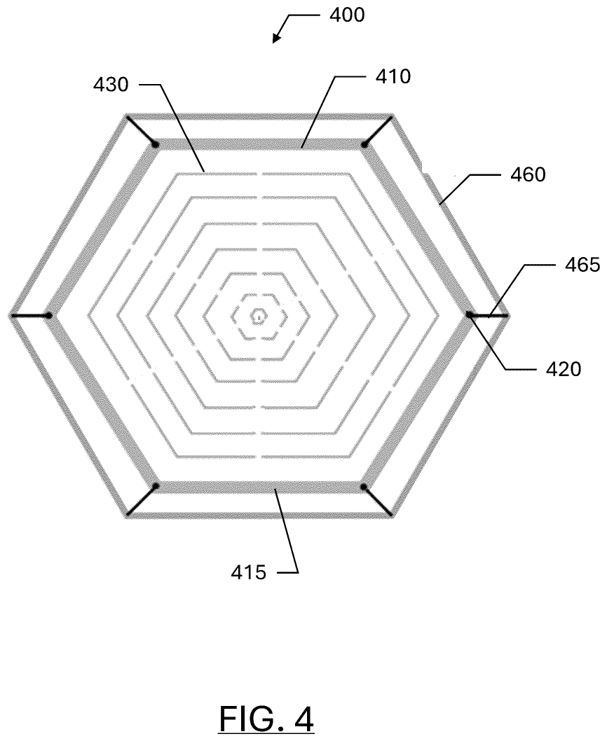

FIG. 4 illustrates a bladder 400 having a hexagon shape suspended within a lightweight frame 460. The bladder 400 includes the perimeter seam 410, the reinforcing strip 415 including grommets 420. The bladder also includes the ribbing 430 to define the internal segments of the bladder. The bladder 400 is secured to the frame 460 through ties 465 between the frame and the grommets 420 of the bladder. These ties could be rigid, semi flexible, or even elastic to some degree depending upon the use case and the need for reduction of any movement of the bladder. The frame 460 can be positioned on legs standing with the bladder arranged horizontally (e.g., with the top and bottom surface substantially horizontal), vertically, or at any angle necessary for the specific use case.

The bladder of example embodiments can contain a type of gas to allow optical detection of the gas in a spectral region of detection defined by the type of gas, comprising a gas bladder configured to contain the gas. The gas container of an example embodiment is formed from a material substantially optically transparent in the spectral region of detection defined by the type of gas. Also part of an example system is a frame for holding the gas container in a position for the optical detection. The gas container is a bladder sealed to prevent the gas from leaking into an environment. In one embodiment, the substantially optically transparent material is at least 90% transparent in the spectral region of detection.

It is contemplated that the gas is detectable in a long-wavelength spectral region, and the material of the gas container bladder is polyethylene. The polyethylene may be an ultra-pure polyethylene. In one configuration, the ultra-pure (polyethylene or other type material) is pure enough to decrease absorptions in the region to less than 15%. In one embodiment, the term ultra-pure is defined as pure enough to decrease absorptions in the region of interest to less than 10%. In one embodiment, the term ultra-pure is defined as pure enough to decrease absorptions in the region of interest to less than 20%. In one embodiment, the term ultra-pure is defined as pure enough to decrease absorptions in the region of interest to less than 5%. In one configuration, the gas is detectable in a short-wavelength spectral region, and the material of the gas container includes high purity perfluorinated hydrocarbons devoid of optical absorptions due to C—H stretching modes.

A blackbody may be disposed adjacent to the gas container. The system may further comprise at least one first temperature measurement device in the gas container to measure a temperature of the gas and at least one second temperature measurement device on the blackbody to measure a temperature of the blackbody. The frame may be configured to position the gas container: 1) in relation to a blackbody positioned adjacent to the gas container; and/or 2) in relation to an illumination source. In one embodiment, one or more sides of the gas container are reflective.

Also disclosed a method of containing a type of gas to allow optical detection of the gas in a spectral region of detection defined by the type of gas, comprising providing a gas container in the form of a bladder configured to contain the gas. The gas container is formed from a material substantially optically transparent in the spectral region of detection defined by the type of gas. This method also includes placing the gas in the gas container and holding the gas container in a position for the optical detection, such as with a frame as depicted in FIG. 4.

In one embodiment, the gas container is a bladder sealed to prevent the gas from leaking into an environment. The container may be formed from the substantially optically transparent material is at least 90% transparent in the spectral region of detection. In one configuration the gas is detectable in a long-wavelength spectral region and the material of the gas container is polyethylene. The polyethylene may be an ultra-pure polyethylene. For example, the ultra-pure polyethylene may be at least 85% pure.

In one configuration, the gas is detectable in a short-wavelength spectral region, and the material of the gas container includes perfluorinated hydrocarbons. It is further contemplated that a polypropylene mesh may be around the gas container to retain the gas container in position. In one embodiment, the method further includes disposing at least one first temperature measurement device in the gas container to measure a temperature of the gas and at least one second temperature measurement device on a blackbody disposed adjacent to the gas container to measure a temperature of the blackbody.

The term “substantially optically transparent” is herein defined to mean at least 85%-95% optically transparent in a given spectral region of detection. In another embodiment, substantially optically transparent is defined to mean greater than 95% optically transparent. In another embodiment, substantially optically transparent is defined to mean from 80% to 85% optically transparent. Substantially optically transparent means that the optical features of the gas are not obscured by optical absorption features of the bladder used to contain the gas. For example, a target gas under test may have a small absorption at 9.5 microns in the long wave infrared spectral region. A bladder optical absorption feature at 12 microns may have little consequence in the application of the technology to that gas detection because the 12 micron absorption feature is outside of the spectral region of interest. This holds true as long as the bladder material is not significantly warmed from an increased temperature of the gas.

Embodiments of the present disclosure may be used for calibrating systems used in remote detection of gases, sensor performance studies and phenomenon research. The remote gas detection systems may be used for detection of chemical weapons, to detect leaks from chemical plants, for detection of greenhouse gases, to detect gases from man-made disasters, and so forth. Such systems typically create optical features to view absorptive or emissive features of the gas under study, from which a type of gas and characteristics of the gas may be determined based on known techniques. The systems usually use either solar illumination of the gas, or an emissive illumination (such as a thermal radiance, a blackbody light source or an artificial light source such as a laser) of the gas. The known techniques can determine a type of gas based on a known “fingerprint” or spectral features associated with each type of gas, which can be compared with the detected optical characteristics of the gas to determine the type of gas and/or properties associated with the gas.

Embodiments of the present invention provide systems and methods for containing a type of gas to allow optical detection of the gas in a spectral region of detection defined by the type of gas, with the gas being contained in a gas container held by a frame. The gas container is formed from a material substantially optically transparent in the spectral region of detection defined by the type of gas. Thus, a different gas container is used for different types of gases when the spectral region of detection defined by the type of gas is different, as further explained herein.

The frame holds the gas container in a position for the optical detection. The frame confines the optical path to a known value and therefore the absorptivity due to Beer's law optical absorption. This bladder dimension defines the optical path (distance, angles) which is a key part of the measured absorption.

The systems and methods of the embodiments of the invention are configured to facilitate optical calibration of the remote gas detection systems by using a known type of gas in the gas container, at a known concentration, and having a known path length. By utilizing a gas container bladder that is substantially optically transparent and positioning the gas container within the frame for proper directed energy, solar illumination or exposure to thermal radiance, optical features of the gas can be imaged through direct transmission of the gas container or projected as spectrally encoded “shadows” onto nearby man-made structures or onto the ground.

A remote gas detection system, such as one flying over in an airplane or other air, land, or space based location, then images the bladder and makes a determination of the type of gas and its path length, which then can be used to calibrate the remote gas detection system by comparing the measured values to the known values and adjusting the system to the known values.

The system of the embodiments of the invention can use one or more thermocouples or any other temperature sensing element in the bladder or part of associate diagnostics attached to the bladder or pointed at the bladder to measure the temperature of the gas. For measurements using differential temperature such as mid- and long wave spectroscopy, one or more thermocouples on a blackbody positioned in a background area (or on the ground) to measure a temperature of the blackbody to measure a temperature difference between the gas and the blackbody. A unique feature of the system is the ability to accurately determine the temperature difference between an emissive background surface behind the container and the temperature of the gas inside the container. As this difference is now controlled, accurate performance determinations are possible. For long wave (8-12 microns), mid-wave (3-5 microns), shortwave (1-2.5 microns), and visible light (0.400-0.900 microns), spectral radiometers can be used to measure the radiance energy reflecting or emitting from the background area. The background area may or may not be an engineered surface.

As illustrated in FIGS. 1-4, embodiments of a system of the present disclosure comprises a gas container in the form of a bladder having spatial dimensions that support the optical requirements of the sensor system under test. An important characteristic of the gas container is that the material of the gas container is selected for its light (energy) transmission properties. In particular, the material of the gas container is selected to be substantially optically transparent in the spectral region of detection defined by the type of gas. This results in the imager being able to see the gas without seeing the bladder because the bladder is substantially optically transparent in the region of detection. Since the absorption spectrum of the bladder material is well known, the residue absorption features of the thin bladder can be removed by any of a number of analytical techniques.

The gas in the bladders can also be imaged in the short wavelength range using the sun as the light source. The sun rays, positioned at an angle to the bladder, will travel through the bladder and leave a shadow on the ground, and in that shadow will be the spectra of the gas, which can be captured by the imager in the remote gas detection system. Additionally, some of the rays from the sun that do not pass through the bladder on their way to the ground will reflect off the ground and back up through the bladder, which will also transmit the spectra of the gas to the imager. The same bladder is thus imaged at two spots in the image. Similarly, light rays that do not interact with the bladder, and the gas therein, are also reflected to the imager to serve as a baseline of the reflection that does not internee with the gas in the bladder.

Certain gases are detected using sensors operating in a short-wave infrared range, while other gases are detected using sensors operating in a long-wave infrared range. This is because different gases have “fingerprints” detectable in these different ranges. Accordingly, the material of the gas container is selected to be substantially optically transparent in the detection range for the particular type of gas in one or more spectral regions. In most passive infrared or short-wave sensor testing scenarios, multiple gases are released for viewing by a remote sensing system in separate or combined releases. By using the containers, a single gas or multiple gases will be viewed across one region such that detection fidelity can be evaluated. Multiple bladders provided discreet concentration levels. In practical applications, different bladder materials will likely be used for thermal versus SWIR/VNIR (short-wave infrared/visible to near infrared) applications.

Gas bladder materials have been chosen to be as chemically and optically as inert as possible. For applications using laser systems as the light source, laser fluence will typically not be high enough to damage the materials. For detection of gases in a long-wavelength range (7.5-13.5 microns), the gas container may be a bladder made from a high-density polyethylene (HDPE) material that is ultra-pure and very thin (less than 2 mil in most cases), to provide a substantially optically transparent container in the long-wavelength region of optical detection. The optical thickness is decreased as much as possible given the environmental deployment. Low wind scenarios can support thinner bladder thicknesses. The high-density polyethylene bladder is formulated to have little or no absorptions in the long-wavelength region. Commercially available polyethylene bags generally have other materials included that have absorptions in the long-wavelength region and are not ultra-pure.

For detection of gases in a short-wavelength range (1-2.5 microns), the gas container may be a bladder made from 100% perfluorinated hydrocarbons (PFC) to provide a substantially optically transparent container in the short-wavelength region of optical detection. The perfluorinated hydrocarbon bladder has little to no absorptions if all C—H stretches in the in the short-wavelength spectral region have been removed. Like the long wave application, a spectral transmission of above 85%-90% is desired from 400-2500 nm for a substantially optically transparent container (bag/bladder/other shape). In another embodiment, substantially optically transparent is defined to mean greater than 90% optically transparent in the spectral range of interest. In another embodiment, substantially optically transparent is defined to mean from 80% to 85% optically transparent in the spectral range of interest. Other materials can be used if the other materials are substantially optically transparent in the region of detection for the particular gas.

The gas container of an example embodiment is attached to a frame as shown in FIG. 4 to be held in place in relation to the illumination or a blackbody source for optical detection and to protect the gas container from the ambient environment. The frame may be made from aluminum, although any other material could also be used that is capable of holding the gas container in place and maintaining a uniform path length. The gas container and frame may be supported above the ground or other surface by a support structure.

The gas container may be a bladder of dimensions one meter by one meter by 30 centimeters thick, for example. Multiple gas containers may be used, such as four bladders positioned together within one or more frames to produce a larger target for illumination, such as a two meter by two meter target for illumination. Any size or shape of container may be used. In one embodiment the thickness of the bladder is uniform across the container.

The frame may be mounted at an elevation and aspect (angles to the sun and ground) to produce uniform optical targets from a distance. This can produce multiple target signatures for solar illumination targets and produce a temperature differential between the gas and the background or blackbody for thermal imaging of the gases. The containers may be placed generally parallel to the ground, generally perpendicular to the ground, or at an angle.

The gas container will often be exposed to high winds in remote test venues. Wind and debris mitigation structures may be used to protect the gas container but are not required in embodiments of the invention. Because the gas container may be an extremely thin bladder as described above, the gas container may be relatively fragile and thus subject to the environmental conditions such as wind. While the gas container is attached to the frame in several locations, to provide further stability to the gas container, a support material 16 in the form of “strings” or mesh may be positioned around the gas container. The mesh material may be polypropylene for long-wave measurements, and perfluorinated plastics for short-wave measurements, with the strings positioned at approximately six inches apart to prevent interference with the imaging, although other spacings could be used.

Embodiments of the invention may use gas cylinders and/or portable gas release control systems to provide initial filling and a method to alter the concentrations of gases in the container. Partial fills of the container with a buffer gas may be used. The bladders may be filled against the frame and the mesh material. The system may also use in-situ optical and chemical diagnostics to monitor concentrations of gas in the gas container, gas temperature and ground temperature. For toxic gases that require remote filling, mass flow meters, valves and the remotely controlled diagnostics are used to fill the bladder containers. Ranges of internal concentration are made using a serial dilution of buffer gas such as nitrogen. An aliquot of gas is placed in the bag/bladder and pure nitrogen gas is added to fill the bladder. To lower the concentration over this maximum, the bladder is partially deflated and more buffer gas is added. For toxic gases being used in a scenario where 0% mixed gas can be released to the environment during filling, a scrubber is used on the outflow to contain the gas that exits the bladder, or the gas is captured into a secure container after the experiment.

The gas container bladder may be monitored with diagnostics that are appropriate for the gases being used. These can include commercial modulated laser interrogators for short-wave gases, modified closed-path FT-IRs for LWIR-active gases, photoionization detectors, electrochemical cells, and gas samplers that can quantify the gas concentration.

The gas in the container may be circulated or stirred to prevent the formation of thermal gradients in the bladder. Any type stirring device, fan, or other gas circulation device may be used to circulate the gas in the gas container. The system may also provide temperature monitoring of the gases and substrates. For example, one or more thermocouples or thermal cameras can be used to monitor the temperatures of the gases and substrates in the system. By measuring the temperature of the gas and substrate temperatures, a quantitative value of 11T may be determined, where

$$11 T = {T}_{gas} - {T}_{background} \qquad ( 1 )$$

The gas container can be loaded with a gas with an absorption or emission in a known spectral range for calibration of the sensor used in the remote sensing platform. For example, the remote sensing platform may be one or more of a long-wave hyperspectral imaging system, a long path infrared spectrometer, or a laser-based system such as a LIDAR.

Spectral information is recorded when there is a difference in the spectral radiance or absorption. For passive hyperspectral applications this is most often caused by a temperature difference between the gas and the background, as set forth above in Equation (1). This temperature or spectral radiance difference between gas and background can be either positive or negative. The temperature difference can be quantified using the gas containers of the invention where a true calibrated temperature difference and/or associated spectral radiance can be measured. This is not possible for open air releases.

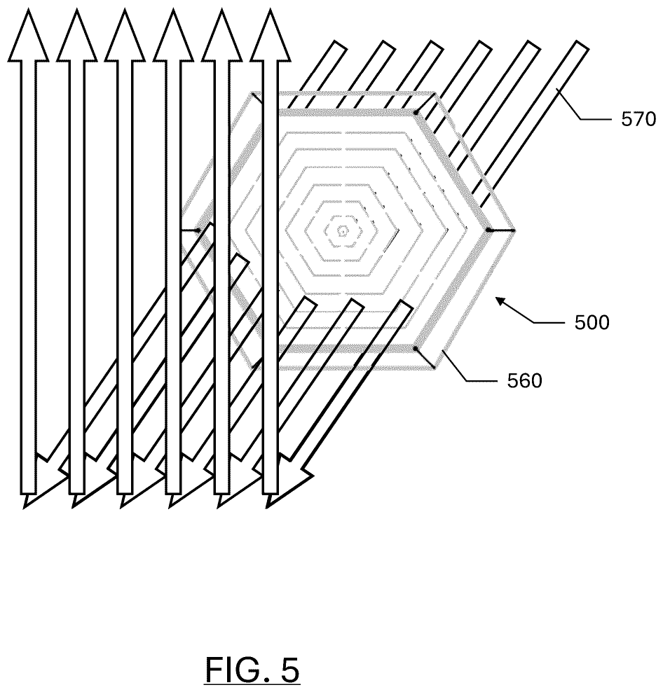

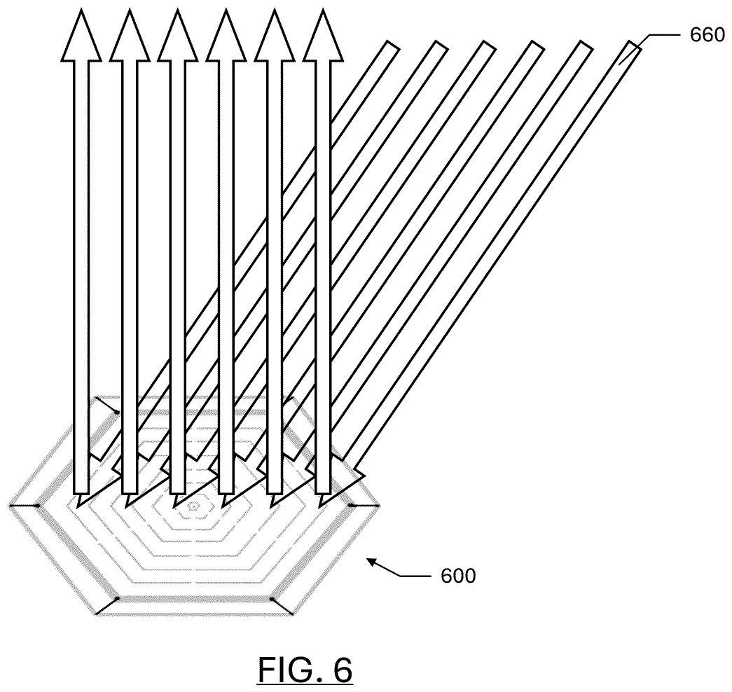

FIGS. 5 and 6 illustrate a gas container bladder 500 and frame 560 where the desired optical measurement is solar illuminated by the sun's rays 570 (other illumination source). For reflectance-based tests, sun angles are calculated and used to derive the bladder position relative to the solar trajectory across the sky. In all cases described herein, the sun is not directly overhead (everywhere except solar noon in the tropics). FIG. 5 shows light rays 570 from the sun passing through the gas container bladder 500, illuminating the ground and scattering towards the remote platform where the sensor is located (such as an airplane).

FIG. 6 shows the light rays 670 that first reflect off the ground, then pass through the gas container bladder 600 and are seen by the remote sensor above. The declination of the sun in the sky allows for the separation of the shadow from the view seen by the sensor of the optical system under test. Simple modeling assures that the shadow is placed such that two distinct optical features track separately during the test period. This produces two side-by-side targets for observation by the sensor of the remote sensing platform, each of which contain the spectral features of the gas imparted by passing through the gas container bladder 600 one time. Reflections which do not pass through the gas container bladder 600 may also be captured.

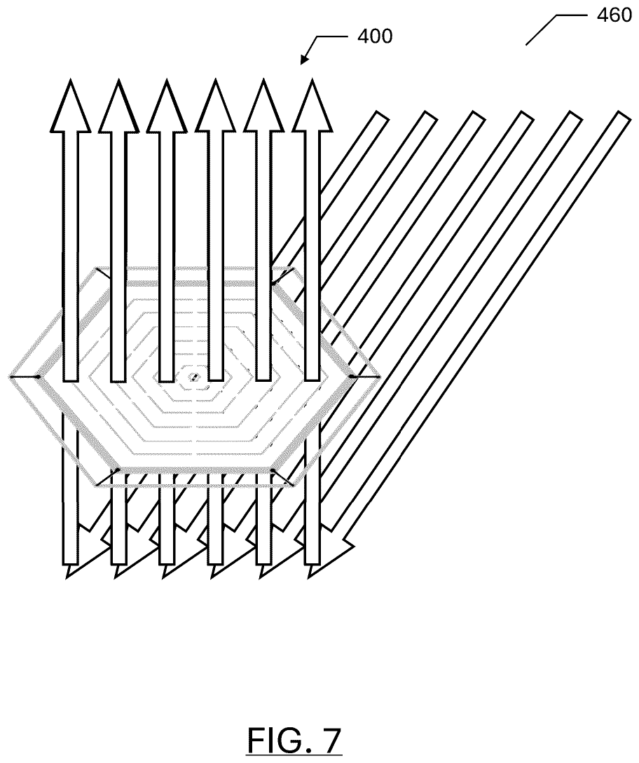

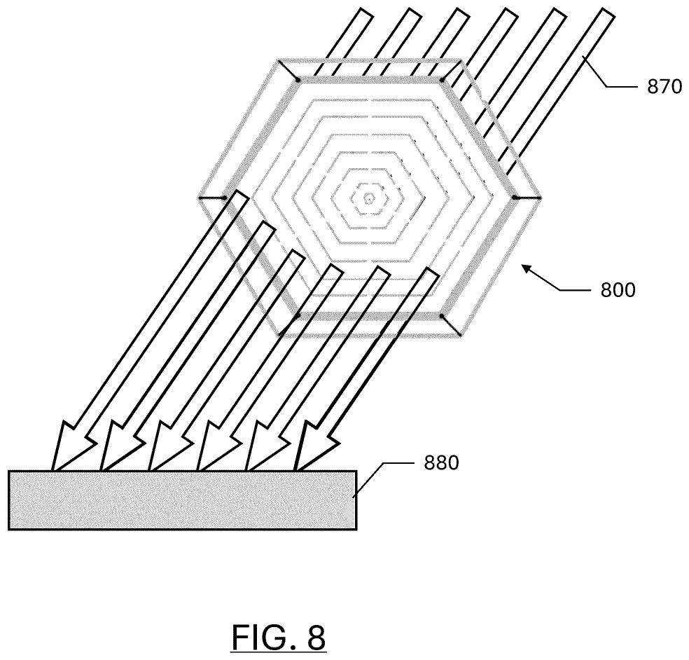

FIG. 7 illustrates an alternative deployment configuration where the gas container bladder 700 is placed on the ground and light passes through the bladder twice. FIG. 8 illustrates another alternative deployment configuration where the gas container bladder 800 is placed at an angle relative to the light rays 870 and used to cast a spectral feature on to a second structure 880, which may be a wall. In this configuration, the spectral information of the gas in the gas container bladder 800 is projected onto the second structure 880. The benefit to this arrangement is that more complex optical effects such as side-welling radiance can be studied by remote sensing researchers.

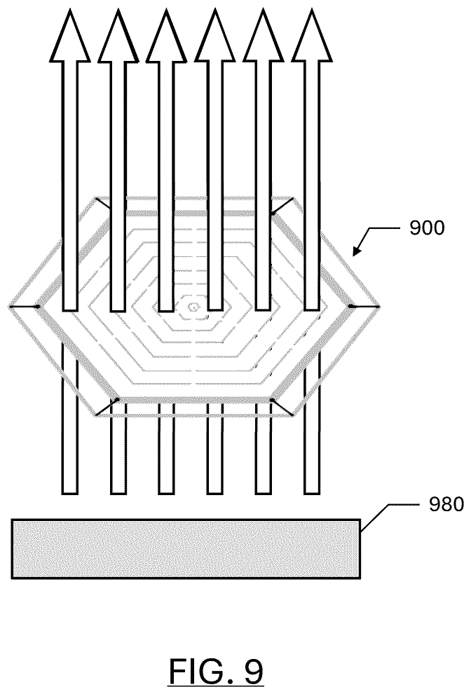

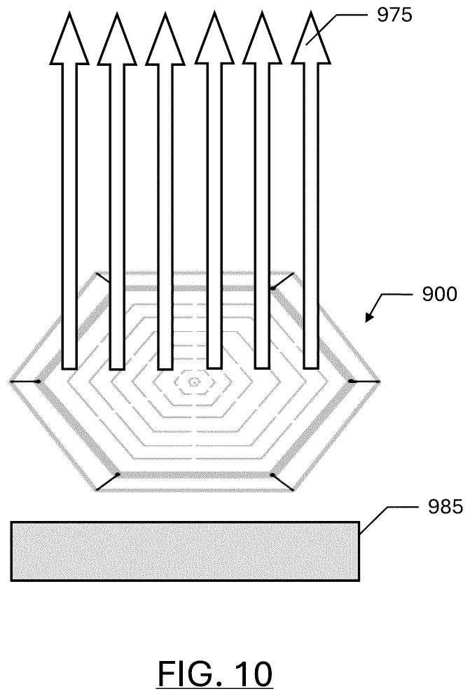

FIGS. 9 and 10 illustrates two geometries where the gas container bladder 900 and its support structure frame 960 can be used to detect changes in emissivity or absorption. In LWIR (Long Wave Infrared) applications, the sky down welling radiance is minimal compared to sunlight in the visible range. Thus, the gas container bladders 900 can be placed near to the ground as there is no shadow cast. As long as there is temperature separation between the ground and the container gas bladder 900, large arrays of bladders can be arrayed to create a large, long wave target. In FIG. 9, the spectral radiant source 980 emits rays 970 that pass through the gas container bladder as an absorber. The remaining radiance is spectral radiance of background radiation minus spectral radiance of the gas and bladder 900 where T_{background} is greater than T_{gas}. In FIG. 10, there is a low radiance background 985 and radiance is emitted from the bladder as an emitter as rays 975, where T_{gas} is significantly greater than T_{background}.

Background reflectivity remains an important factor that can produce a wide range of radiance effects for researchers. For example, an unpainted aluminum background can create a mirror-like surface in LWIR. Under clear skies, the aluminum surface can appear to have the temperature of the “cold-of-space” if the image sensor “sees” the sky as the reflection. This creates the situation where the target above the background is seen as an emission (see FIG. 10).

Conditions where the background and the gas have similar spectral temperatures can be complex. By measuring the background and gas container temperatures, the present disclosure allows for creation of conditions where there is always sufficient 11T for calibration. Embodiments of the present disclosure allow for measuring the temperature of the gas and the blackbody so that a more accurate estimation of 11T is obtained as compared to open air releases. Conductive heating of the background blackbody allows for measuring sensor sensitivity for different gases and conditions.

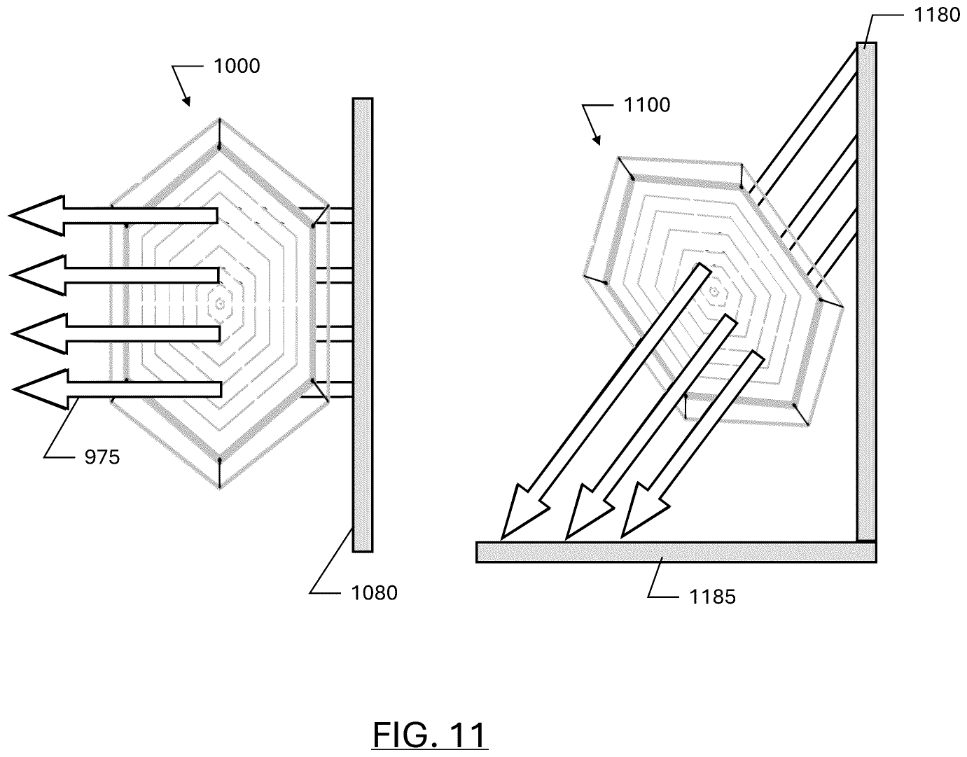

FIG. 11 illustrates alternative arrangements for the gas container bladders 1000 and 1100, including a spectral radiant sources 1080 and 1180 positioned adjacent to the gas container bladders 1000 and 1100. As is the case in FIG. 8, complex optical geometries can be explored and allow for more algorithm research on side-welling and other optical effects that complicate gas concentration quantification. Gas is the absorber in both cases, with T_{gas} is less than T_{background} for gas container bladder 1000. For gas container bladder 1100, the ground surface 1185 has observed ground surface at T_{ground}, where T_{gas} T_{background} is significantly greater than T_{gas} and T_{ground}, where the radiance reaching the ground surface is spectral radiance of the background minus the spectral radiance of the gas.

In one embodiment of the disclosure, multiple gas containers bladders can be used simultaneously to image multiple bladders in one or more imaging runs. For example, if it is desired to perform a calibration of a remote gas detection system for one type of gas at numerous different concentrations, this can be done by placing different concentrations in the gas container bladder and performing repeated imaging runs to compare the results to the known type of gas and concentration, or by using multiple gas containers and doing one imaging run with a separate image of each gas container that is individually compared to the known values. Any number of gas container bladders could be used.

Additionally, multiple different types of gas could be calibrated for during one or more imaging runs using a plurality of the gas container bladders, with each gas container containing a different type of gas and comparing the measured results to the known types of gas. Further, these methods could be combined with multiple gas container bladders with some of the gas container bladders having varying types of gas and varying concentrations. These methods have the advantage of reducing the calibration time and the cost of planes flying over.

The gas container bladders could also be arranged in a vertically stacked arrangement, with a gas bladder having one type of gas being disposed over a gas bladder having another type of gas. Furthermore, other factors concerning the gas in the gas container may be altered, such as the humidity in the bladder to simulate real world environments for phenomenon study. Also, gases could be mixed in the bladder to cause reactions which could be studied by imaging the bladder to study the resulting spectra.

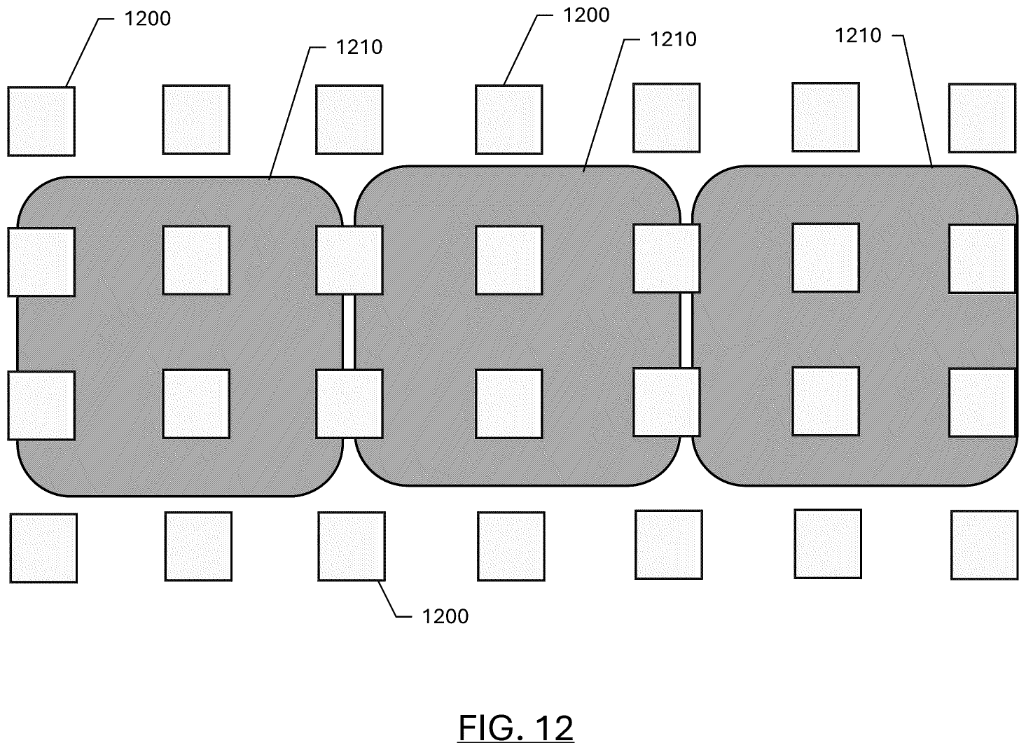

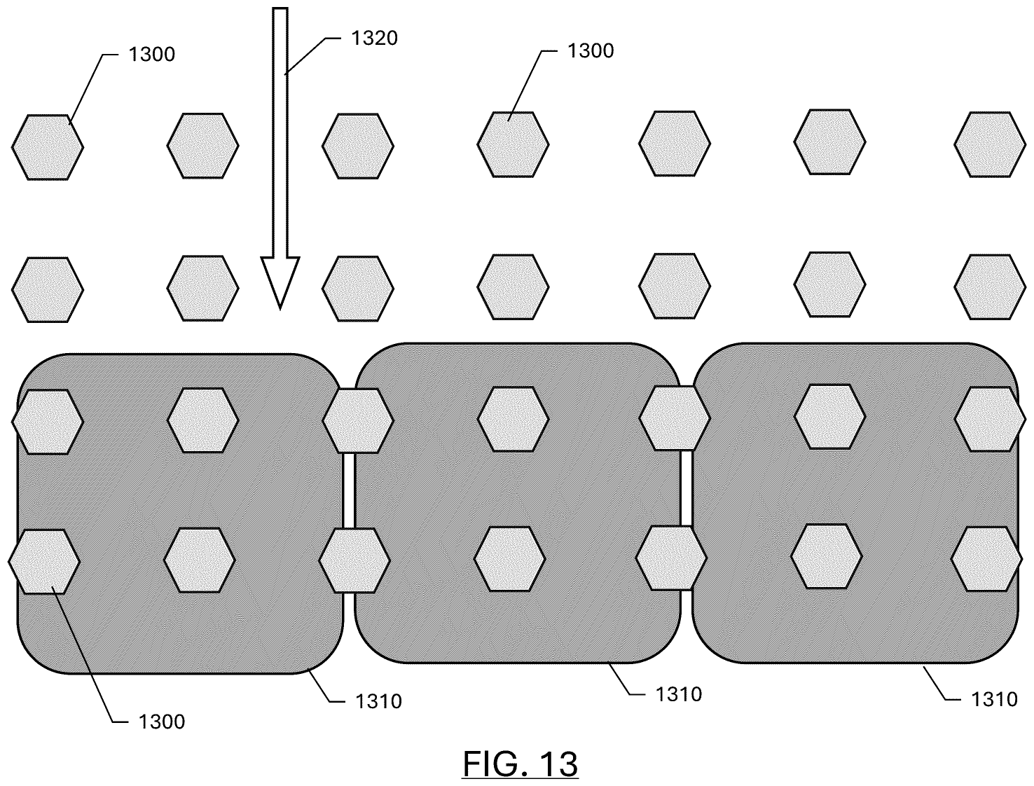

FIGS. 12-13 illustrate possible geometries for using different bladder sizes and shapes. The figures create a sense of the logistics required to adequately cover the target pixel size. In this exercise, the remote projected pixel is illustrated as 1210. The example pixel 1210 is eight meters on a side or an area of about 64 square meters. Percent pixel coverage is important for fixed imagers as the mismatch can lead to dilution of the signal. For some gases, the concentration and path length cannot be increased to overcome pixel dilution and there will be minimum values for percent pixel coverage depending on the gas absorption coefficient.

FIG. 12 illustrates the bladder geometry for 28 targets or bladders 1200 that are each two meters by two meters. According to the illustrated embodiment, 28 targets is the minimum number possible with an aligned geometry where the targets are set to a fixed sensor-to-target rotation. Embodiments described herein are uniquely configured to be able to provide the requisite 28 bladders 1200 as two-by-two meter square bladders, which is not feasible in prior embodiments.

FIG. 13 illustrates an embodiment using hexagonal bladders 1300. The hexagonal shape is easier to stabilize with a frame, such as an aluminum frame. However, the area is smaller with a 1-meter apothem length. The illustrated geometry calls for 28 two-meter wide bladders 1300 in a 4×7 array as shown. This area of targets is sufficient if the array is aligned with the flight line 1320 as shown.

Many modifications and other embodiments of the disclosure set forth herein will come to mind to one skilled in the art to which these embodiments pertain having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A system for containing a type of gas to allow optical detection of the gas in a spectral region of detection defined by the type of gas, comprising:
a top layer of material;
a bottom layer of material, wherein the top layer is sealed to the bottom layer forming a bladder with a perimeter seam and forming a cavity;
ribbing between the top layer of material and the bottom layer of material defining segments of the cavity; and
a frame, wherein the frame supports the bladder.

2. The system of claim 1, further comprising a reinforcing strip substantially covering the perimeter seam.

3. The system of claim 2, further comprising one or more grommets within the reinforcing strip, wherein the bladder is attached to the frame using the one or more grommets.

4. The system of claim 1, wherein the segments of the cavity are of substantially similar height.

5. The system of claim 1, wherein the bladder comprises a rectangular shape.

6. The system of claim 1, wherein the bladder comprises a hexagonal shape.

7. The system of claim 1, wherein at least one of the top layer of material and the bottom layer of material are substantially optically transparent material of at least 80% transparent in a spectral region of detection for the system.

8. The system of claim 1, wherein at least one of the top layer of material and the bottom layer of material is sufficiently pure to decrease absorptions in a spectral region of detection for the system to less than between 5% and 20%.

9. The system of claim 8, wherein the at least one of the top layer of material and the bottom layer of material is polyethylene.

10. The system of claim 1, wherein the bladder is formed on a roll, and wherein the bladder is cut to a user-defined length at a cut line, and wherein the cut line is sealed to close the cavity.

11. A method of containing a type of gas to allow optical detection of the gas in a spectral region of detection defined by the type of gas, the method comprising:
filling a bladder with the gas, wherein the bladder comprises:
a top layer of material;
a bottom layer of material, wherein the top layer is sealed to the bottom layer forming a bladder with a perimeter seam and forming a cavity;
ribbing between the top layer of material and the bottom layer of material defining segments of the cavity; and
supporting the bladder on a frame.

12. The method of claim 11, wherein the bladder comprises a reinforcing strip substantially covering the perimeter seam.

13. The method of claim 12, wherein the reinforcing strip further comprises one or more grommets within the reinforcing strip, wherein supporting the bladder on the frame comprises attaching the bladder to the frame using the one or more grommets.

14. The method of claim 11, wherein the segments of the cavity are of substantially similar height.

15. The method of claim 11, wherein the bladder comprises a rectangular shape.

16. The method of claim 11, wherein the bladder comprises a hexagonal shape.

17. The method of claim 11, wherein at least one of the top layer of material and the bottom layer of material are substantially optically transparent material of at least 80% transparent in the spectral region of detection.

18. The method of claim 11, wherein at least one of the top layer of material and the bottom layer of material is sufficiently pure to decrease absorptions in the spectral region of detection to less than between 5% and 20%.

19. The method of claim 18, wherein the at least one of the top layer of material and the bottom layer of material is polyethylene.

20. The method of claim 11, further comprising:
forming the bladder on a roll;
cutting the bladder to a user-defined length at a cut line; and
sealing the bladder at the cut line to form the cavity.
